(19) United States
(12) Patent Application Publication
(10) Pub. No.: US 2018/0036289 A1
(43) Pub. Date: Feb. 8, 2018

(54) COBICISTAT FOR USE IN CANCER TREATMENTS

(71) Applicant: Gilead Sciences, Inc., Foster City, CA (US)

(72) Inventors: Ana Zurisadai Gonzalez Buenrostro, San Mateo, CA (US); Bernard Patrick Murray, Foster City, CA (US); Richard Michael Neve, San Mateo, CA (US); Lianhong Xu, Palo Alto, CA (US)

(21) Appl. No.: 15/666,417

(22) Filed: Aug. 1, 2017

Related U.S. Application Data
(60) Provisional application No. 62/371,105, filed on Aug. 4, 2016.

(51) Int. Cl. A61K 31/427; A61K 31/5377; A61K 31/675; A61K 31/513

(52) U.S. Cl. CPC A61K 31/427; A61K 31/513; A61K 31/5377; A61K 31/675

(57) ABSTRACT

The disclosure describes methods for and compositions for treatment of patients having cancers expressing CYP3A enzymes by co-administration of cobicistat with an anticancer agent.

### CROSS REFERENCE TO RELATED APPLICATIONS

This non-provisional application claims the benefit of Provisional Application No. 62/371,105, filed Aug. 4, 2016, which is incorporated herein by reference.

### FIELD

Described herein are methods and uses for treating patients suffering from cancers expressing a CPY3A enzyme by co-administration of a selective CYP3A inhibitor with an anticancer agent.

### BACKGROUND

Cobicistat is a CYP3A inhibitor used in combination therapy for treatment of HIV. Cobicistat is described in WO 2008/010921, incorporated herein by reference. Because there are a growing number of patients with lack of sensitivity to anticancer agents, a need exists for treatment regimens which can enhance the efficacy of existing treatments.

### SUMMARY

One embodiment of the present invention provides a method of treating a patient suffering from cancer, comprising administered cobicistat and an anticancer agent. In a particular embodiment, the cancer and/or anticancer agent is described below.

Another embodiment provides a method for treating a patient suffering from cancer comprising administering to said patient: (a) an anticancer agent; and (b) cobicistat; wherein, the cancer comprises cells expressing a CYP3A enzyme and the concentration of the anticancer agent in the cells is increased after administration of cobicistat. In another embodiment, the cancer comprises cells overexpressing a CYP3A enzyme.

Another embodiment provides a method for enhancing the effect of an anticancer agent in a patient suffering from cancer comprising administering to said patient: (a) the anticancer agent; and (b) cobicistat; wherein, the cancer comprises cells expressing a CYP3A enzyme and the effect of the anticancer agent in the cells is increased after administration of cobicistat. In another embodiment, the cancer comprises cells overexpressing a CYP3A enzyme.

Another embodiment provides a method for reducing metabolism of an anticancer agent in a patient suffering from cancer comprising administering to said patient: (a) the anticancer agent; and (b) cobicistat; wherein, the cancer comprises cells expressing a CYP3A enzyme and the metabolism of the anticancer agent in the cells is decreased after administration of cobicistat. In another embodiment, the cancer comprises cells that overexpress the CYP3A enzyme.

Another embodiment of the invention provides for a method for increasing sensitivity to an anticancer agent in a patient suffering from cancer comprising administering to said patient: (a) the anticancer agent; and (b) cobicistat; wherein, cobicistat increases sensitivity to the anticancer agent.

Another embodiment provides a pharmaceutical composition comprising (a) an anticancer agent; (b) cobicistat; and (c) a carrier.

### BRIEF DESCRIPTION OF THE FIGURES

FIG. 1 depicts the relative CYP3A4 expression in normal tissue (top bars) versus the expression of CYP3A4 in tumors (lower bars). The expression profiles vary the greatest in tumors, with particular tumors overexpressing CYP3A4 (e.g. the “dots” depicted for colon rectum adenocarcinoma). In some of the tumors (e.g. pancreatic adenocarcinoma) the majority of the tumors overexpressed CYP3A4 as compared to the normal cell lines.

FIG. 2 depicts the relative CYP3A5 expression in normal tissue (top bars) versus the expression of CYP3A5 in tumors (lower bars). As with CYP3A4, the expression profiles vary the greatest in tumors, with particular tumors overexpressing CYP3A5 (e.g. the bar extending to the right of the tumor levels for cervical squamous cell carcinoma and endocervical adenocarcinoma). In some of the cell lines (e.g. kidney cancers) the majority of tumors overexpressed CYP3A5 as compared to the normal cell lines.

### DETAILED DESCRIPTION

The following definitions are used throughout the specification:

“Anticancer agent” refers to an agent capable of treating or preventing cancer. A list of anticancer agents for use herein is provided below. It is understood that reference to an “anticancer agent” includes one or more different anticancer agents.

“Cobicistat” refers to 1,3-thiazol-5-ylmethyl (2R,5R)-(5-{[(2S)-2-[(methyl{[2-(propan-2-yl)-1,3-thiazol-4-yl]methyl}carbamoyl)amino]]-4-(morpholin-4-yl)butanamido}-1,6-diphenylhexan-2-yl)carbamate) and has been shown to be a mechanism-based inhibitor of CYP3A enzymes, CYP3A4 and CYP3A5, with greater specificity than ritonavir. Xu et al., ACS Med. Chem. Lett. (2010), 1, pp. 209-13. The structure of cobicistat is shown below, as Formula Ia:

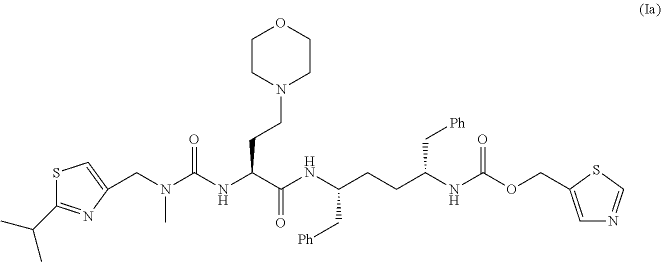 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

As used herein, the term “co-administer” refers to administration of two or more agents within a 24 hour period of each other, for example, as part of a clinical treatment regimen. In other embodiments, “co-administer” refers to administration of two or more agents within 2 hours of each other. In other embodiments, “co-administer” refers to administration of two or more agents within 30 minutes of each other. In other embodiments, “co-administer” refers to administration of two or more agents within 15 minutes of each other. In other embodiments, “co-administer” refers to administration at the same time, either as part of a single formulation or as multiple formulations that are administered by the same or different routes.

“IC₉₅” or “EC₉₅” refers to the inhibitory concentration required to achieve 95% of the maximum desired effect, which in the case of an anticancer agent is the inhibition of cancer cell lines or enzymes implicated in the target cancer (e.g. kinase activity). This value is obtained using an in vitro assay evaluating the concentration-dependent inhibition of cancer cell lines expressing the target or recombinant protein (e.g. a kinase).

“Increasing sensitivity to an anticancer agent by X-fold” refers to the ability of cobicistat to increase the desired effect of the anticancer agent (e.g. IC₅₀ or other metric of efficacy) by X-fold as compared to administration of the anticancer in the absence of cobicistat. Preferably, the “X-fold” is 2-fold, or 1.5-fold, or 3-fold or even 5-fold.

“TI” or “therapeutic index” as used herein refers to the ratio between the median effective dose for the unboosted therapy (ED_{50-U}) and the median effective dose of the anticancer agent when co-administered with cobicistat (ED_{50-cobi}). Consequently, drugs that exhibit a TI of 1 or less present no benefit from cobicistat co-administration. The dosing regimen provided herein provides a TI greater than 1 for the anticancer agent.

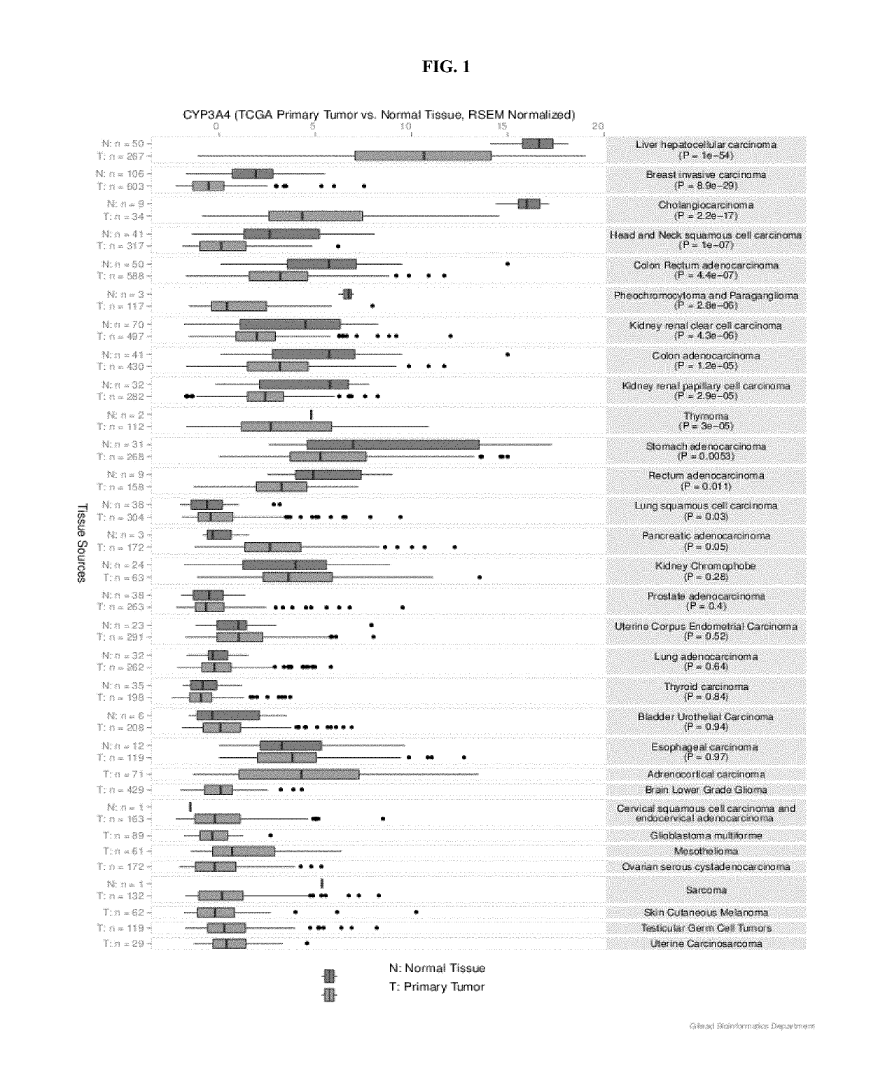

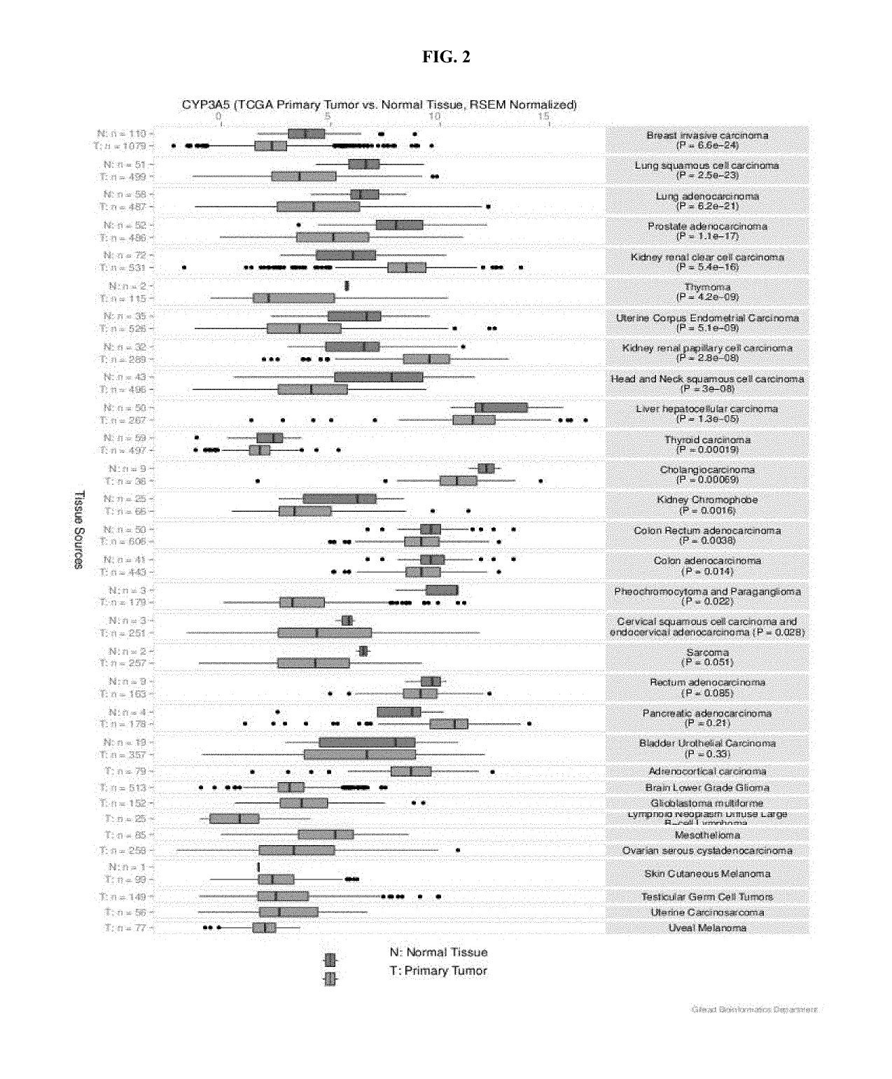

“Overexpression” of a CYP3A enzyme (e.g. CYP3A4 and/or CY3A5, as used herein, refers to the expression of the particular CYP3A enzyme at a level greater in the tumor or cancer cell line as compared to the normal tissue or normal cell line. The expression profiles for various cell lines are depicted in FIG. 1 and FIG. 2. Overexpression can be determined either through biopsy/testing of cell lines to determine the expression level as compared to the standard levels known for the cell line (e.g. those reflected in FIGS. 1 and 2 and known in the art). It is understood that overexpression/overexpressing is encompassed by expression/expressing. Expression or expressing CYP3A as used herein indicates the presence of CYP3A in cells, which can be inhibited by cobicistat.

“Therapeutically effective amount” refers to that amount of the compound being administered which will prevent a condition, or will relieve to some extent one or more of the symptoms of the disorder being treated. Pharmaceutical compositions suitable for use herein include compositions wherein the active ingredients are contained in an amount sufficient to achieve the intended purpose. Determination of a therapeutically effective amount is well within the capability of those skilled in the art, especially in light of the detailed disclosure provided herein. As used herein, treatment refers to inhibition, reduction, elimination or alleviation of a disease as well as prevention.

The present invention also provides a method for the treatment or prophylaxis of diseases, disorders, and conditions. An example of a disease, disorder, or condition includes, but is not limited to, cancer, or a disease, disorder, or condition associated with a cancer.

The active agents, including cobicistat and/or anticancer agents may be administered to a human in any conventional manner. While it is possible for the active agents to be administered as compounds, they are preferably administered as a pharmaceutical composition. The salt, carrier, or diluent should be acceptable in the sense of being compatible with the other ingredients and not deleterious to the recipient thereof. Examples of carriers or diluents for oral administration include cornstarch, lactose, magnesium stearate, talc, microcrystalline cellulose, stearic acid, povidone, crospovidone, dibasic calcium phosphate, sodium starch glycolate, hydroxypropyl cellulose (e.g., low substituted hydroxypropyl cellulose), hydroxypropylmethyl cellulose (e.g., hydroxypropylmethyl cellulose 2910), and sodium lauryl sulfate.

The pharmaceutical compositions may be prepared by any suitable method, such as those methods well known in the art of pharmacy, for example, methods such as those described in Gennaro et al., Remington's Pharmaceutical Sciences (18th ed., Mack Publishing Co., 1990), especially Part 8: Pharmaceutical Preparations and their Manufacture. Such methods include the step of bringing into association the compounds with the carrier or diluent and optionally one or more accessory ingredients. Such accessory ingredients include those conventional in the art, such as, fillers, binders, excipients, disintegrants, lubricants, colorants, flavoring agents, sweeteners, preservatives (e.g., antimicrobial preservatives), suspending agents, thickening agents, emulsifying agents, and/or wetting agents.

In practice, the amount of each compound (e.g. the compounds described herein) to be administered ranges from about 0.001 to 100 mg per kg of body weight, such total dose being given at one time or in divided doses. Each compound may be administered alone or in combination with one or more other drugs (e.g. the compounds and combinations disclosed herein). Preferably, cobicistat is administered QD at 150 mg. Generally, each compound will be administered as a formulation in association with one or more pharmaceutically acceptable excipients. The choice of excipient will to a large extent depend on factors such as the particular mode of administration, the effect of the excipient on solubility and stability, and the nature of the dosage form.

Pharmaceutical compositions suitable for the delivery of compounds described herein and methods for their preparation will be readily apparent to those skilled in the art. Such compositions and methods for their preparation may be found, for example, in Remington's Pharmaceutical Sciences, 19th Edition (Mack Publishing Company, 1995).

### Anticancer Agents:

As described herein, cobicistat is used or combined with one or more anticancer agent, which includes: a chemotherapeutic agent, an anticancer agent, an anti-angiogenic agent, an anti-fibrotic agent, an immunotherapeutic agent, a therapeutic antibody, a bispecific antibody and “antibody-like” therapeutic protein (such as DARTs®, Duobodies®, Bites®, XmAbs®, TandAbs®, Fab derivatives), an antibody-drug conjugate (ADC), a radiotherapeutic agent, an anti-neoplastic agent, an anti-proliferation agent, an oncolytic virus, gene modifiers or editors such as CRISPR (including CRISPR Cas9), zinc finger nucleases or synthetic nucleases (TALENs), a CAR (chimeric antigen receptor) T-cell immunotherapeutic agent, or any combination thereof. These anticancer agents may be in the forms of compounds, antibodies, polypeptides, or polynucleotides. In one embodiment, the application provides a product comprising cobicistat and an additional anticancer agent as a combined preparation for simultaneous, separate, or sequential use in therapy, e.g. a method of treating a disease, disorder, or condition that is mediated by PI3K isoforms.

It is understood that none of the descriptions of anticancer agents includes cobicistat itself.

By way of example, anticancer agents include, inter cal/a, any of the following: 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids and ZD1839 or a pharmaceutically acceptable salt thereof. Additional agents and groupings are discussed further below.

### Targets

The anticancer agents include, but are not limited to, an inhibitor, agonist, antagonist, ligand, modulator, stimulator, blocker, activator or suppressor of a gene, ligand, receptor, protein, factor such as:

adenosine receptor (such as A2B, A2a, A3), Abelson murine leukemia viral oncogene homolog 1 gene (ABL, such as ABL1), Acetyl-CoA carboxylase (such as ACC1/2), adrenocorticotropic hormone receptor (ACTH), activated CDC kinase (ACK, such as ACK1), Adenosine deaminase, Adenylate cyclase, ADP ribosyl cyclase-1, Aerolysin, Angiotensinogen (AGT) gene, murine thymoma viral oncogene homolog 1 (AKT) protein kinase (such as AKT1, AKT2, AKT3), AKT1 gene, Alkaline phosphatase, Alpha 1 adrenoceptor, Alpha 2 adrenoceptor, Alpha-ketoglutarate dehydrogenase (KGDH), Aminopeptidase N, Arginine deiminase, Beta adrenoceptor, Anaplastic lymphoma kinase receptor, anaplastic lymphoma kinase (ALK, such as ALK1), Alk-5 protein kinase, AMP activated protein kinase, Androgen receptor, Angiopoietin (such as ligand-1, ligand-2), apolipoprotein A-I (APOA1) gene, apoptosis signal-regulating kinase (ASK, such as ASK1), Apoptosis inducing factor, apoptosis protein (such as 1, 2), Arginase (I), asparaginase, Asteroid homolog 1 (ASTE1) gene, ataxia telangiectasia and Rad 3 related (ATR) serine/threonine protein kinase, Axl tyrosine kinase receptor, Aromatase, Aurora protein kinase (such as 1, 2), Basigin, BCR (breakpoint cluster region) protein and gene, B-cell lymphoma 2 (BCL2) gene, Bc12 protein, Bc12 binding component 3, BCL2L11 gene, Baculoviral IAP repeat containing 5 (BIRCS) gene, B-Raf proto-oncogene (BRAF), Brc-Abl tyrosine kinase, Beta-catenin, B-lymphocyte antigen CD19, B-lymphocyte antigen CD20, B-lymphocyte stimulator ligand, B-lymphocyte cell adhesion molecule, Bone morphogenetic protein-10 ligand, Bone morphogenetic protein-9 ligand modulator, Brachyury protein, Bradykinin receptor, Bruton's tyrosine kinase (BTK), Bromodomain and external domain (BET) bromodomain containing protein (such as BRD2, BRD3, BRD4), Calmodulin, calmodulin-dependent protein kinase (CaMK, such as CAMKII), Cancer testis antigen 2, Cancer testis antigen NY-ESO-1, Cannabinoid receptor (such as CB1, CB2), Carbonic anhydrase, caspase 8 apoptosis-related cysteine peptidase CASP8-FADD-like regulator, Caspase (such as caspase-3, caspase-7, Caspase-9), Caspase recruitment domain protein-15, Cathepsin G, chemokine (C-C motif) receptor (such as CCR2, CCR4, CCR5), CCR5 gene, Chemokine CC21 ligand, cluster of differentiation (CD) such as CD4, CD27, CD29, CD30, CD33, CD37, CD40, CD40 ligand receptor, CD40 ligand, CD40LG gene, CD44, CD45, CD47, CD49b, CD51, CD52, CD55, CD58, CD66e, CD70 gene, CD74, CD79, CD79b, CD79B gene, CD80, CD95, CD99, CD117, CD122, CDw123, CD134, CDw137, CD158a, CD158b1, CD158b2, CD223, CD276 antigen; Chorionic gonadotropin, Cyclin G1, Cyclin D1, cyclin-dependent kinases (CDK, such as CDK1, CDK1B, CDK2-9), casein kinase (CK, such as CM, CMI), c-Kit (tyrosine-protein kinase Kit or CD117), c-Met (hepatocyte growth factor receptor (HGFR)), CDK-activating kinase (CAK), Checkpoint kinase (such as CHK1, CHK2), Cholecystokinin CCK2 receptor, Claudin (such as 6, 18), Clusterin, Complement C3, COP9 signalosome subunit 5, CSF-1 (colony-stimulating factor 1 receptor), CSF2 gene, clusterin (CLU) gene, Connective tissue growth factor, cyclooxygenase (such as 1, 2), cancer/testis antigen 1B (CTAG1) gene, CTLA-4 (cytotoxic T-lymphocyte protein 4) receptor, CYP2B1 gene, Cysteine palmitoyltransferase porcupine, cytokine signalling-1, cytokine signalling-3, Cytochrome P450 11B2, Cytochrome P450 reductase, cytochrome P450 3A4, cytochrome P450 17A1, Cytochrome P450 17, Cytochrome P450 2D6, (provided they anticancer or cytrochrom modifying agents are something other than cobicistat), Cytoplasmic isocitrate dehydrogenase, Cytosine deaminase, cytosine DNA methyltransferase, cytotoxic T-lymphocyte protein-4, chemokine (C—X—C motif) receptor (such as CXCR4, CXCR1 and CXCR2), Delta-like protein ligand (such as 3, 4), Deoxyribonuclease, Dickkopf-1 ligand, Dihydropyrimidine dehydrogenase, DNA binding protein (such as HU-beta), DNA dependent protein kinase, DNA gyrase, DNA methyltransferase, DNA polymerase (such as alpha), DNA primase, discoidin domain receptor (DDR, such as DDR1), DDR2 gene, dihydrofolate reductase (DHFR), Dipeptidyl peptidase IV, L-dopachrome tautomerase, dUTP pyrophosphatase, echinoderm microtubule like protein 4, epidermal growth factor receptor (EGFR) gene, EGFR tyrosine kinase receptor, Eukaryotic translation initiation factor 5A (EIFSA) gene, Elastase, Elongation factor 1 alpha 2, Elongation factor 2, Endoglin, Endonuclease, Endoplasmin, Endosialin, Endostatin, endothelin (such as ET-A, ET-B), Enhancer of zeste homolog 2 (EZH2), epidermal growth factor, epidermal growth factor receptors (EGFR), Epithelial cell adhesion molecule (EpCAM), Ephrin (EPH) tyrosine kinase (such as Epha3, Ephb4), Ephrin B2 ligand, Epigen, Erb-b2 (v-erb-b2 avian erythroblastic leukemia viral oncogene homolog 2) tyrosine kinase receptor, Erb-b3 tyrosine kinase receptor, Erb-b4 tyrosine kinase receptor, Extracellular signal-regulated kinases (ERK), E-selectin, Estradiol 17 beta dehydrogenase, Estrogen receptor (such as alpha, beta), Estrogen related receptor, Exportin 1, Extracellular signal related kinase (such as 1, 2), Factor (such as Xa, VIIa), Fas ligand, Fatty acid synthase, Ferritin, focal adhesion kinase (FAK, such as FAK2), fibroblast growth factor (FGF, such as FGF1, FGF2, FGF4), FGF-2 ligand, FGF-5 ligand, Fibronectin, Fms-related tyrosine kinase 3 (Flt3), farnesoid x receptor (FXR), Folate, Folate transporter 1, Folate receptor (such as alpha), folate hydrolase prostate-specific membrane antigen 1 (FOLH1), paired basic amino acid cleaving enzyme (FURIN), FYN tyrosine kinase, Galactosyltransferase, Galectin-3, glucocorticoid-induced TNFR-related protein GITR receptor, Glucocorticoid, Beta-glucuronidase, Glutamate carboxypeptidase II, glutaminase, Glutathione S-transferase P, Glypican 3 (GPC3), glycogen synthase kinase (GSK, such as 3-beta), Granulocyte-colony stimulating factor (GCSF) ligand, Granulocyte macrophage colony stimulating factor (GM-CSF) receptor, gonadotropin-releaseing hormone (GNRH), growth factor receptor-bound protein 2 (GRB2), molecular chaperone groEL2 gene, Grp78 (78 kDa glucose-regulated protein) calcium binding protein, Imprinted Maternally Expressed Transcript (H19) gene, Heat stable enterotoxin receptor, Heparanase, Hepatocyte growth factor, Heat shock protein gene, Heat shock protein (such as 27, 70, 90 alpha, beta), Hedgehog protein, HERV-H LTR associating protein 2, Hexose kinase, tyrosine-protein kinase HCK, Histamine H2 receptor, histone deacetylase (HDAC, such as 1, 2, 3, 6, 10, 11), Histone H1, Histone H3, Histone methyltransferase (DOT1L), Human leukocyte antigen (HLA), HLA class I antigen (A-2 alpha), HLA class II antigen, Homeobox protein NANOG, mitogen-activated protein kinase kinase kinase kinase 1 (MAP4K1, HPK1), HSPB1 gene, Human papillomavirus (such as E6, E7) protein, Hyaluronidase, Hyaluronic acid, Hypoxia inducible factor-1 alpha, Intercellular adhesion molecule 1 (ICAM-1), immunoglobulin (such as G, G1, G2, K, M), indoleamine 2,3-dioxygenase (IDO, such as IDO1), indoleamine pyrrole 2,3-dioxygenase 1 inhibitor, I-Kappa-B kinase (IKK, such as IKKβε), Immunoglobulin Fc receptor, Immunoglobulin gamma Fc receptor (such as I, III, IIIA), Interleukin 1 ligand, interleukin 2 ligand, Interleukin-2, IL-2 gene, IL-1 alpha, IL-1 beta, IL-2, IL-2 receptor alpha subunit, IL-3 receptor, IL-4, IL-6, IL-7, IL-8, IL-12, IL-15, IL-12 gene, IL-17, Interleukin 13 receptor alpha 2, Interleukin-29 ligand, interleukin-1 receptor-associated kinase 4 (IRAK4), Insulin-like growth factor (such as 1, 2), insulin receptor, Integrin alpha-V/beta-3, Integrin alpha-V/beta-5, Integrin alpha-V/beta-6, Integrin alpha-5/beta-1, Integrin alpha-4/beta-1, integrin alpha-4/beta-7, Interferon inducible protein absent in melanoma 2 (AIM2), interferon (such as alpha, alpha 2, beta, gamma), interferon type I receptor, isocitrate dehydrogenase (such as IDH1, IDH2), Janus kinase (JAK, such as JAK1, JAK2), Jun N terminal kinase, Kinase insert domain receptor (KDR), Killer cell Ig like receptor, Kisspeptin (KISS-1) receptor, v-kit Hardy-Zuckerman 4 feline sarcoma viral oncogene homolog (KIT) tyrosine kinase, KIT gene, Kinesin-like protein KIF11, kallikrein-related peptidase 3 (KLK3) gene, Kirsten rat sarcoma viral oncogene homolog (KRAS) gene, lactoferrin, lymphocyte activation gene 3 protein (LAG-3), lysosomal-associated membrane protein family (LAMP) gene, Lanosterol-14 demethylase, LDL receptor related protein-1, Leukotriene A4 hydrolase, Listeriolysin, L-Selectin, Luteinizing hormone receptor, Lyase, Lymphocyte antigen 75, lysine demethylases (such as KDM1, KDM2, KDM4, KDM5, KDM6, A/B/C/D), Lymphocyte function antigen-3 receptor, lymphocyte-specific protein tyrosine kinase (LCK), Lymphotactin, Lyn (Lck/Yes novel) tyrosine kinase, Lysophosphatidate-1 receptor, lysyl oxidase protein (LOX), lysyl oxidase-like protein (LOXL, such as LOXL2), Lysyl oxidase homolog 2, Macrophage migration inhibitory fact, melanoma antigen family A3 (MAGEA3) gene, MAGEC1 gene, MAGEC2 gene, Major vault protein, myristoylated alanine-rich protein kinase C substrate (MARCKS) protein, Melan-A (MART-1) melanoma antigen, Mas-related G-protein coupled receptor, matrix metalloprotease (MMP, such as MMP2, MMP9), myeloid cell leukemia 1 (MCL1) gene, Mcl-1 differentiation protein, macrophage colony-stimulating factor (MCSF) ligand, Melanoma associated antigen (such as 1, 2, 3, 6), melanocyte stimulating hormone ligand, Melanocyte protein Pmel 17, Membrane copper amine oxidase, Mesothelin, Metabotropic glutamate receptor 1, mitogen-activated protein kinase (MEK, such as MEK1, MEK2), Hepatocyte growth factor receptor (MET) gene, MET tyrosine kinase, methionine aminopeptidase-2, mitogen-activate protein kinase (MAPK), Mdm2 p53-binding protein, Mdm4 protein, Metalloreductase STEAP1 (six transmembrane epithelial antigen of the prostate 1), Metastin, Methyltransferase, Mitochondrial 3 ketoacyl CoA thiolase, MAPK-activated protein kinase (such as MK2), mTOR (mechanistic target of rapamycin (serine/threonine kinase), mTOR complex (such as 1, 2), mucin (such as 1, 5A, 16), mut T homolog (MTH, such as MTH1), Myc proto-oncogene protein, NAD ADP ribosyltransferase, natriuretic peptide receptor C, Neural cell adhesion molecule 1, Neurokinin receptor, Neuropilin 2, Nitric oxide synthase, Nuclear Factor (NF) kappa B, NF kappa B activating protein, Neurokinin 1 (NK1) receptor, NK cell receptor, NK3 receptor, NKG2 A B activating NK receptor, NIMA-related kinase 9 (NEK9), Noradrenaline transporter, Notch (such as Notch-2 receptor, Notch-3 receptor), nucleophosmin-anaplastic lymphoma kinase (NPM-ALK), 2,5-oligoadenylate synthetase, Nuclear erythroid 2-related factor 2, Nucleolin, Nucleophosmin, O-methylguanine DNA methyltransferase, Ornithine decarboxylase, Orotate phosphoribosyltransferase, orphan nuclear hormone receptor NR4A1, Opioid receptor (such as delta), Osteocalcin, Osteoclast differentiation factor, Osteopontin, OX-40 (tumor necrosis factor receptor superfamily member 4 TNFRSF4, or CD134) receptor, 2 oxoglutarate dehydrogenase, purinergic receptor P2X ligand gated ion channel 7 (P2X7), Parathyroid hormone ligand, p53 tumor suppressor protein, P3 protein, Programmed cell death 1 (PD-1), Proto-oncogene serine/threonine-protein kinase (PIM, such as PIM-1, PIM-2, PIM-3), Poly ADP ribose polymerase (PARP, such as PARP1, 2 and 3), p38 kinase, p38 MAP kinase, platelet-derived growth factor (PDGF, such as alpha, beta), P-Glycoprotein (such as 1), Platelet-derived growth factor (PDGF, such as alpha, beta), PKN3 gene, P-Selectin, phosphatidylinositol 3-kinase (PI3K), phosphoinositide-3 kinase (PI3K such as alpha, delta, gamma), phosphorylase kinase (PK), placenta growth factor, Pleiotropic drug resistance transporter, Plexin B1, Polo-like kinase 1, peroxisome proliferator-activated receptors (PPAR, such as alpha, delta, gamma), Preferentially expressed antigen in melanoma (PRAME) gene, Probable transcription factor PML, Programmed cell death ligand 1 inhibitor (PD-L1), Progesterone receptor, prostate specific antigen, Prostatic acid phosphatase, Prostanoid receptor (EP4), proteasome, Protein farnesyltransferase, protein kinase (PK, such as A, B, C), Protein E7, protein tyrosine kinase, Protein tyrosine phosphatase beta, polo-like kinase (PLK), PLK1 gene, Prenyl-binding protein (PrPB), protoporphyrinogen oxidase, Prosaposin (PSAP) gene, phosphatase and tensin homolog (PTEN), Purine nucleoside phosphorylase, Pyruvate kinase (PYK), Pyruvate dehydrogenase (PDH), Pyruvate dehydrogenase kinase, Raf protein kinase (such as 1, B), RAF1 gene, Ras GTPase, Ras gene, 5-Alpha-reductase, RET gene, Ret tyrosine kinase receptor, retinoblastoma associated protein, retinoic acid receptor (such as gamma), Retinoid X receptor, Rheb (Ras homolog enriched in brain) GTPase, Rho (Ras homolog) associated protein kinase 2, ribonuclease, Ribonucleotide reductase (such as M2 subunit), Ribosomal protein S6 kinase, RNA polymerase (such as I, II), Ron (Recepteur d'Origine Nantais) tyrosine kinase, ROS1 (ROS proto-oncogene 1, receptor tyrosine kinase) gene, Ros1 tyrosine kinase, Runt-related transcription factor 3, 5100 calcium binding protein A9, Sarco endoplasmic calcium ATPase, Gamma-secretase, Secreted frizzled related protein-2, Semaphorin-4D, SL cytokine ligand, Serine protease, Signaling lymphocytic activation molecule (SLAM) family member 7, spleen tyrosine kinase (SYK), Src tyrosine kinase, tumor progression locus 2 (TPL2), serine/threonine kinase (STK), signal transduction and transcription (STAT, such as STAT-1, STAT-3, STAT-5), Second mitochondria-derived activator of caspases (SMAC) protein, smoothened (SMO) receptor, Sodium phosphate cotransporter 2B, Sodium iodide cotransporter, Somatostatin receptor (such as 1, 2, 3, 4, 5), Sonic hedgehog protein, Specific protein 1 (Sp1) transcription factor, Sphingomyelin synthase, Sphingosine-1-phosphate receptor-1, Sphingosine kinase (such as 1, 2), SRC gene, STAT3 gene, six-transmembrane epithelial antigen of the prostate (STEAP) gene, Steroid sulfatase, stimulator of interferon genes protein, Stimulator of interferon genes (STING) receptor, Stromal cell-derived factor 1 ligand, SUMO (small ubiquitin-like modifier), Superoxide dismutase, Survivin protein, Synapsin 3, Syndecan-1, Synuclein alpha, serine/threonine-protein kinase (TBK, such as TBK1), TATA box-binding protein-associated factor RNA polymerase I subunit B (TAF1B) gene, T-cell surface glycoprotein CD8, T-cell CD3 glycoprotein zeta chain, T-cell differentiation antigen CD6, T cell surface glycoprotein CD28, Tec protein tyrosine kinase, Tek tyrosine kinase receptor, telomerase, Tenascin, Telomerase reverse transcriptase (TERT) gene, Transforming growth factor (TGF, such as beta) kinase, TGF beta 2 ligand, T-cell immunoglobulin and mucin-domain containing-3 (TIM-3), Tissue factor, Tumor necrosis factor (TNF, such as alpha, beta), TNF related apoptosis inducing ligand, TNFR1 associated death domain protein, TNFSF9 gene, TNFSF11 gene, trophoblast glycoprotein (TPBG) gene, Transferrin, Tropomyosin receptor kinase (Trk) receptor (such as TrkA, TrkB, TrkC), Trophoblast glycoprotein, Thymidylate synthase, Tyrosine kinase with immunoglobulin-like and EGF-like domains (TIE) receptor, Toll-like receptor (TLR such as 1-13), topoisomerase (such as I, II, III), Tumor protein 53 (TP53) gene, Transcription factor, Transferase, Transforming growth factor TGF-β receptor kinase, Transglutaminase, Translocation associated protein, Transmembrane glycoprotein NMB, Tumor necrosis factor 13C receptor, Thymidine kinase, Thymidine phosphorylase, Thymidylate synthase, Thymosin (such as alpha 1), Thyroid hormone receptor, Trop-2 calcium signal transducer, Thyroid stimulating hormone receptor, Tryptophan 5-hydroxylase, Tyrosinase, tyrosine kinase (TK), Tyrosine kinase receptor, Tyrosine protein kinase ABL1 inhibitor, tank-binding kinase (TBK), Thrombopoietin receptor, TNF-related apoptosis-inducing ligand (TRAIL) receptor, Tubulin, Tumor suppressor candidate 2 (TUSC2) gene, Tyrosine hydroxylase, Ubiquitin-conjugating enzyme E2I (UBE2I, UBC9), Ubiquitin, Ubiquitin carboxyl hydrolase isozyme L5, Ubiquitin thioesterase-14, Urease, Urokinase plasminogen activator, Uteroglobin, Vanilloid VR1, Vascular cell adhesion protein 1, vascular endothelial growth factor receptor (VEGFR), V-domain Ig suppressor of T-cell activation (VISTA), VEGF-1 receptor, VEGF-2 receptor, VEGF-3 receptor, VEGF-A, VEGF-B, Vimentin, Vitamin D3 receptor, Proto-oncogene tyrosine-protein kinase Yes, Wee-1 protein kinase, Wilms' tumor protein, Wilms' tumor antigen 1, X-linked inhibitor of apoptosis protein, Zinc finger protein transcription factor or any combination thereof.

As used herein, the term “chemotherapeutic agent” or “chemotherapeutic” (or “chemotherapy” in the case of treatment with a chemotherapeutic agent) is meant to encompass any non-proteinaceous (i.e., non-peptidic) chemical compound useful in the treatment of cancer.

### Mechanism of Action

The anticancer agent includes agents defined by their mechanism of action or class, including:
anti-metabolites/anti-cancer agents such as pyrimidine analogs floxuridine, capecitabine, cytarabine, CPX-351 (liposomal cytarabine, daunorubicin), TAS-118;
purine analogs, folate antagonists (such as pralatrexate), and related inhibitors;
antiproliferative/antimitotic agents including natural products such as vinca alkaloid (vinblastine, vincristine) and microtubule such as taxane (paclitaxel, docetaxel), vinblastin, nocodazole, epothilones, vinorelbine) (NAVELBINE®), and epipodophyllotoxins (etoposide, teniposide);
DNA damaging agents such as actinomycin, amsacrine, busulfan, carboplatin, chlorambucil, cisplatin, cyclophosphamide) (CYTOXAN®), dactinomycin, daunorubicin, doxorubicin, epirubicin, iphosphamide, melphalan, merchlorethamine, mitomycin C, mitoxantrone, nitrosourea, procarbazine, taxol, Taxotere, teniposide, etoposide, and triethylenethiophosphoramide;
DNA-hypomethylating agent such as guadecitabine (SGI-110)
antibiotics such as dactinomycin, daunorubicin, doxorubicin, idarubicin, anthracyclines, mitoxantrone, bleomycins, plicamycin (mithramycin), and;
enzymes such as L-asparaginase which systemically metabolizes L-asparagine and deprives cells which do not have the capacity to synthesize their own asparagine;
antiplatelet agents;
a DNAi oligonucleotide targeting Bcl-2 such as PNT2258;
agents that activate or reactivate latent human immunodeficiency virus (HIV) such as panobinostat or romidepsin
asparaginase stimulators, such as crisantaspase (Erwinase®) and GRASPA (ERY-001, ERY-ASP);
pan-Trk, ROS1 and ALK inhibitors such as entrectinib
anaplastic lymphoma kinase (ALK) inhibitors such as alectinib
antiproliferative/antimitotic alkylating agents such as nitrogen mustards cyclophosphamide and analogs (melphalan, chlorambucil, hexamethylmelamine, and thiotepa), alkyl nitrosoureas (carmustine) and analogs, streptozocin, and triazenes (dacarbazine);
antiproliferative/antimitotic antimetabolites such as folic acid analogs (methotrexate);
platinum coordination complexes (cisplatin, oxiloplatinim, and carboplatin), procarbazine, hydroxyurea, mitotane, and aminoglutethimide;
hormones, hormone analogs (estrogen, tamoxifen, goserelin, bicalutamide, and nilutamide), and aromatase inhibitors (letrozole and anastrozole);
anticoagulants such as heparin, synthetic heparin salts, and other inhibitors of thrombin;
fibrinolytic agents such as tissue plasminogen activator, streptokinase, urokinase, aspirin, dipyridamole, ticlopidine, and clopidogrel;
antimigratory agents;
antisecretory agents (breveldin);
immunosuppressives tacrolimus, sirolimus, azathioprine, and mycophenolate;
compounds (TNP-470, genistein) and growth factor inhibitors (vascular endothelial growth factor inhibitors, and
fibroblast growth factor inhibitors such as FPA14;
angiotensin receptor blockers, nitric oxide donors;
antisense oligonucleotides, such as AEG35156;
DNA interference oligonucleotides, such as PNT2258, AZD-9150
antibodies such as trastuzumab and rituximab;
anti-HER3 antibodies, such as LJM716
anti-HER2 antibodies such as margetuximab
anti-HLA-DR antibodies such as IMMU-114
anti-IL-3 antibodies, such as JNJ-56022473
anti-OX40 antibodies such as MEDI6469
anti-EphA3 antibodies, such as KB-004
an anti-CD20 antibody such as obinutuzumab
an anti-programmed cell death protein 1 (anti-PD-1) antibody such as nivolumab (OPDIVO®, BMS-936558, MDX-1106), pembrolizumab (KEYTRUDA®, MK-3477, SCH-900475, lambrolizumab, CAS Reg. No. 1374853-91-4), pidilizumab, and anti-programmed death-ligand 1 (anti-PD-L1) antibodies such as BMS-936559, atezolizumab (MPDL3280A), durvalumab (MEDI4736), avelumab (MSB0010718C), and MDX1105-01
CXCR4 antagonists such as BL-8040
CXCR2 antagonist such as AZD-5069
GM-CSF antibodies such as lenzilumab
Selective estrogen receptor downregulator (SERD) such as fulvestrant (Faslodex®)
a transforming growth factor-beta (TGF-beta) kinase antagonist such as galunisertib
a bispecific antibody such as MM-141 (IGF-1/ErbB3), MM-111 (Erb2/Erb3), JNJ-64052781 (CD19/CD3)
Mutant selective EGFR inhibitors, such as PF-06747775, EGF816, ASP8273, ACEA-0010, BI-1482694
Alpha-ketoglutarate dehydrogenase (KGDH) inhibitors such as CPI-613
XPO1 inhibitors such as selinexor (KPT-330)
Isocitrate dehydrogenase 2 (IDH2) inhibitors such as enasidenib (AG-221), and IDH1 inhibitors such as AG-120, and AG-881 (IDH1 and IDH2).
Agents that target the interleukin-3 receptor (IL-3R) such as SL-401
Arginine deiminase stimulators, such as pegargiminase (ADI-PEG-20)
antibody-drug conjugates, such as MLN0264 (anti-GCC, guanylyl cyclase C), T-DM1 (trastuzumab emtansine, Kadcycla), milatuzumab-doxorubicin (hCD74-DOX), brentuximab vedotin, DCDT2980S, polatuzumab vedotin, SGN-CD70A, SGN-CD19A, inotuzumab ozogamicin, lorvotuzumab mertansine, SAR3419, isactuzumab govitecan
anti-claudin-18.2 antibodies such as IMAB362
β-catenin inhibitors, such as CWP-291
a CD73 antagonist such as MEDI-9447;
c-PIM inhibitors, such as PIM447
a BRAF inhibitor such as dabrafenib, vemurafenib
a sphingosine kinase-2 (SK2) inhibitor such as Yeliva® (ABC294640)
cell cycle inhibitors such as selumetinib (MEK1/2), sapacitabine
AKT inhibitors such as MK-2206, ipatasertib, afuresertib
anti-CTLA-4 (cytotoxic T-lymphocyte protein-4) inhibitor such as tremelimumab
c-MET inhibitors, such as AMG-337, savolitinib, tivantinib (ARQ-197), capmatinib, tepotinib
inhibitors of CSF1R/KIT and FLT3 such as PLX3397
a kinase inhibitor such as vandetanib;
E selectin antagonists such as GMI-1271
differentiation inducers such as tretinoin;
epidermal growth factor receptor (EGFR) inhibitors such as osimertinib (AZD-9291)
topoisomerase inhibitors (doxorubicin, daunorubicin, dactinomycin, eniposide, epirubicin, etoposide, idarubicin, irinotecan, mitoxantrone, pixantrone, sobuzoxane, topotecan, and irinotecan, MM-398 (liposomal irinotecan), vosaroxin and corticosteroids (cortisone, dexamethasone, hydrocortisone, methylprednisolone, prednisone, and prednisolone);
growth factor signal transduction kinase inhibitors;
dysfunction inducers;
nucleoside analogs such as DFP-10917
Axl inhibitors such as BGB-324
BET inhibitors such as INCB-054329, add Gilead's compound
PARP inhibitors such as olaparib, rucaparib, veliparib
Proteasome inhibitors such as ixazomib, carfilzomib (Kyprolis®)
Glutaminase inhibitors such as CB-839
vaccines such as peptide vaccine TG-01 (RAS), bacterial vector vaccines such as CRS-207/GVAX, autologous Gp96 vaccine, dendritic cells vaccines, Oncoquest-L vaccine, DPX-Survivac, ProstAtak, DCVAC, ADXS31-142
anti-cancer stem cells, such as demcizumab (anti-DLL4, Delta-like ligand 4, Notch pathway), napabucasin (BBI-608)
smoothened (SMO) receptor inhibitors, such as Odomzo® (sonidegib, formerly LDE-225), LEQ506, vismodegib (GDC-0449), BMS-833923, glasdegib (PF-04449913), LY2940680, and itraconazole;
interferon alpha ligand modulators, such as interferon alfa-2b, interferon alpha-2a biosimilar (Biogenomics), ropeginterferon alfa-2b (AOP-2014, P-1101, PEG IFN alpha-2b), Multiferon (Alfanative, Viragen), interferon alpha 1b, Roferon-A (Canferon, Ro-25-3036), interferon alfa-2a follow-on biologic (Biosidus) (Inmutag, Inter 2A), interferon alfa-2b follow-on biologic (Biosidus-Bioferon, Citopheron, Ganapar) (Beijing Kawin Technology-Kaferon) (AXXO-interferon alfa-2b), Alfaferone, pegylated interferon alpha-1b, peginterferon alfa-2b follow-on biologic (Amega), recombinant human interferon alpha-1b, recombinant human interferon alpha-2a, recombinant human interferon alpha-2b, veltuzumab-IFN alpha 2b conjugate, Dynavax (SD-101), and interferon alfa-n1 (Humoferon, SM-10500, Sumiferon);
interferon gamma ligand modulators, such as interferon gamma (OH-6000, Ogamma 100);
IL-6 receptor modulators, such as tocilizumab, siltuximab, AS-101 (CB-06-02, IVX-Q-101);
Telomerase modulators, such as tertomotide (GV-1001, HR-2802, Riavax) and imetelstat (GRN-163, JNJ-63935937)
DNA methyltransferases inhibitors, such as temozolomide (CCRG-81045), decitabine, guadecitabine (S-110, SGI-110), KRX-0402, and azacitidine;
DNA gyrase inhibitors, such as pixantrone and sobuzoxane;
Bcl-2 family protein inhibitor ABT-263, venetoclax (ABT-199), ABT-737, and AT-101;
Notch inhibitors such as LY3039478, tarextumab (anti-Notch2/3), BMS-906024
anti-myostatin inhibitors such as landogrozumab
hyaluronidase stimulators such as PEGPH-20
Wnt pathway inhibitors such as SM-04755, PRI-724
gamma-secretase inhibitors such as PF-03084014
IDO inhibitors such as indoximod
Grb-2 (growth factor receptor bound protein-2) inhibitor BP1001 (liposomal Grb-2)
TRAIL pathway-inducing compounds, such as ONC201
Focal adhesion kinase inhibitors such as VS-4718, defactinib
hedgehog inhibitors such as saridegib, sonidegib (LDE225), glasdegib and vismodegib
Aurora kinase inhibitors such as alisertib (MLN-8237)
modulators of HSPB1 activity (heat shock protein 27, HSP27), such as brivudine, apatorsen
ATR inhibitor such as AZD6738, and VX-970
mTOR inhibitors, such as sapanisertib
Hsp90 inhibitors such as AUY922
Murine double minute (mdm2) oncogene inhibitors such as DS-3032b
CD137 agonist such as urelumab
Anti-KIR monoclonal antibodies such as lirilumab (IPH-2102)
Antigen CD19 inhibitors such as MOR208, MEDI-551, AFM-11
CD44 binders such as A6
CYP17 inhibitors, such as VT-464, ASN-001, ODM-204.
RXR agonists such as IRX4204
TLRs (Toll-like receptors) agonists such as IMO-8400
A hedgehog/smoothened (hh/Smo) antagonist such as taladegib
Immunomodulators such as complement C3 modulators, such as Imprime PGG
Intratumural immune-oncology agents such as G100 (TLR4 agonist)
IL-15 agonists such as ALT-803
EZH2 (enhancer of zeste homolog 2) inhibitors such as tazemetostat
Oncolytic viruses, such as pelareorep, and talimogene laherparepvec)
DOT1L (histone methyltransferase) inhibitors such as pinometostat (EPZ-5676)
toxins such as Cholera toxin, ricin, Pseudomonas exotoxin, Bordetella pertussis adenylate cyclase toxin, diphtheria toxin, and caspase activators;
and chromatin.
DNA plasmid such as BC-819
PLK inhibitors of PLK 1, 2, and 3, such as volasertib (PLK1).
Apoptosis Signal-Regulating Kinase (ASK) Inhibitors: ASK inhibitors include ASK1 inhibitors. Examples of ASK1 inhibitors include, but are not limited to, those described in WO 2011/008709 (Gilead Sciences) and WO 2013/112741 (Gilead Sciences).
Bruton's Tyrosine Kinase (BTK) Inhibitors: Examples of BTK inhibitors include, but are not limited to, (S)-6-amino-9-(1-(but-2-ynoyl)pyrrolidin-3-yl)-7-(4-phenoxyphenyl)-7H-purin-8(9H)-one, acalabrutinib (ACP-196), BGB-3111, HM71224, ibrutinib, M-2951, ONO-4059, PRN-1008, spebrutinib (CC-292), TAK-020.
Cyclin-dependent Kinase (CDK) Inhibitors: CDK inhibitors include inhibitors of CDK 1, 2, 3, 4, 6 and 9, such as abemaciclib, alvocidib (HMR-1275, flavopiridol), AT-7519, FLX-925, LEE001, palbociclib, ribociclib, rigosertib, selinexor, UCN-01, and TG-02.
Discoidin Domain Receptor (DDR) Inhibitors: DDR inhibitors include inhibitors of DDR1 and/or DDR2. Examples of DDR inhibitors include, but are not limited to, those disclosed in WO 2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO 2013/034933 (Imperial Innovations).
Histone Deacetylase (HDAC) Inhibitors: Examples of HDAC inhibitors include, but are not limited to, abexinostat, ACY-241, AR-42, BEBT-908, belinostat, CKD-581, CS-055 (HBI-8000), CUDC-907, entinostat, givinostat, mocetinostat, panobinostat, pracinostat, quisinostat (JNJ-26481585), resminostat, ricolinostat, SHP-141, valproic acid (VAL-001), vorinostat.
Janus Kinase (JAK) Inhibitors: JAK inhibitors inhibit JAK1, JAK2, and/or
JAK3. Examples of JAK inhibitors include, but are not limited to, AT9283, AZD1480, baricitinib, BMS-911543, fedratinib, filgotinib (GLPG0634), gandotinib (LY2784544), INCB039110, lestaurtinib, momelotinib (CYT0387), NS-018, pacritinib (SB1518), peficitinib (ASP015K), ruxolitinib, tofacitinib (formerly tasocitinib), and XL019.
Lysyl Oxidase-Like Protein (LOXL) Inhibitors: LOXL inhibitors include inhibitors of LOXL1, LOXL2, LOXL3, LOXL4, and/or LOXL5. Examples of LOXL inhibitors include, but are not limited to, the antibodies described in WO 2009/017833 (Arresto Biosciences). Examples of LOXL2 inhibitors include, but are not limited to, the antibodies described in WO 2009/017833 (Arresto Biosciences), WO 2009/035791 (Arresto Biosciences), and WO 2011/097513 (Gilead Biologics).
Matrix Metalloprotease (MMP) Inhibitors: MMP inhibitors include inhibitors of MMP1 through 10. Examples of MMP9 inhibitors include, but are not limited to, marimastat (BB-2516), cipemastat (Ro 32-3555) and those described in WO 2012/027721 (Gilead Biologics).
Mitogen-activated Protein Kinase (MEK) Inhibitors: MEK inhibitors include antroquinonol, binimetinib, cobimetinib (GDC-0973, XL-518), MT-144, selumetinib (AZD6244), sorafenib, trametinib (GSK1120212), uprosertib+trametinib.
Phosphatidylinositol 3-kinase (PI3K) Inhibitors: PI3K inhibitors include inhibitors of PI3Kγ, PI3Kδ, PI3β, PI3Kα, and/or pan-PI3K. Examples of PI3K inhibitors include, but are not limited to, ACP-319, AEZA-129, AMG-319, AS252424, BAY 10824391, BEZ235, buparlisib (BKM120), BYL719 (alpelisib), CH5132799, copanlisib (BAY 80-6946), duvelisib, GDC-0941, GDC-0980, GSK2636771, GSK2269557, idelalisib (Zydelig®), IPI-145, IPI-443, KAR4141, LY294002, Ly-3023414, MLN1117, OXY111A, PA799, PX-866, RG7604, rigosertib, RP5090, taselisib, TG100115, TGR-1202, TGX221, WX-037, X-339, X-414, XL147 (SAR245408), XL499, XL756, wortmannin, ZSTK474, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences).
Spleen Tyrosine Kinase (SYK) Inhibitors: Examples of SYK inhibitors include, but are not limited to, 6-(1H-indazol-6-yl)-N-(4-morpholinophenyl)imidazo[1,2-alpyrazin-8-amine, BAY-61-3606, cerdulatinib (PRT-062607), entospletinib, fostamatinib (R788), HMPL-523, NVP-QAB 205 AA, R112, R343, tamatinib (R406), and those described in U.S. Pat. No. 8,450,321 (Gilead Conn.). and those described in U.S. 2015/0175616.
Tyrosine-kinase Inhibitors (TKIs): TKIs may target epidermal growth factor receptors (EGFRs) and receptors for fibroblast growth factor (FGF), platelet-derived growth factor (PDGF), and vascular endothelial growth factor (VEGF). Examples of TKIs include, but are not limited to, afatinib, bosutinib, brigatinib, cabozantinib, crenolanib, dacomitinib, dasatinib, dovitinib, E-6201, erlotinib, gefitinib, gilteritinib (ASP-2215), HM61713, icotinib, imatinib, KX2-391 (Src), lapatinib, lestaurtinib, midostaurin, nintedanib, osimertinib (AZD-9291), ponatinib, poziotinib, quizartinib, radotinib, rociletinib, sunitinib, and TH-4000.
Further anticancer agents include: alkylating agents such as thiotepa and cyclophosphamide (CYTOXAN®); alkyl sulfonates such as busulfan, improsulfan, and piposulfan; aziridines such as benzodepa, carboquone, meturedepa, and uredepa; ethylenimines and methylamelamines including altretamine, triethylenemelamine, triethylenephosphoramide, triethylenethiophosphoramide, and trimemylolomelamine; acetogenins, especially bullatacin and bullatacinone; a camptothecin, including synthetic analog topotecan; bryostatin, callystatin; CC-1065, including its adozelesin, carzelesin, and bizelesin synthetic analogs; cryptophycins, particularly cryptophycin 1 and cryptophycin 8; dolastatin; duocarmycin, including the synthetic analogs KW-2189 and CBI-TMI; eleutherobin; 5-azacytidine; pancratistatin; a sarcodictyin; spongistatin; nitrogen mustards such as chlorambucil, chlomaphazine, cyclophosphamide, glufosfamide, evofosfamide, bendamustine, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammaII and calicheamicin phiI1), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogs such as demopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replinishers such as frolinic acid; radiotherapeutic agents such as Radium-223; trichothecenes, especially T-2 toxin, verracurin A, roridin A, and anguidine; taxoids such as paclitaxel) (TAXOL®), abraxane, docetaxel) (TAXOTERE®), cabazitaxel, BIND-014; platinum analogs such as cisplatin and carboplatin, NC-6004 nanoplatin; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; trabectedin, triaziquone; 2,2′,2″-tricUorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine) (GEMZAR®); 6-thioguanine; mercaptopurine; methotrexate; vinblastine; platinum; etoposide (VP-16); ifosfamide; mitroxantrone; vancristine; vinorelbine) (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; FOLFIRI (fluorouracil, leucovorin, and irinotecan); and pharmaceutically acceptable salts, acids, or derivatives of any of the above.

### Anti-Hormonal Agents

Also included in the definition of anticancer agents are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors.

Examples of anti-estrogens and SERMs include, for example, tamoxifen (including NOLVADEX™), raloxifene, droloxifene, 4-hydroxytamoxifen, trioxifene, keoxifene, LY117018, onapristone, and toremifene) (FARESTON®).

Inhibitors of the enzyme aromatase regulate estrogen production in the adrenal glands. Examples include 4(5)-imidazoles, aminoglutethimide, megestrol acetate) (MEGACE®), exemestane, formestane, fadrozole, vorozole) (RIVISOR®), letrozole) (FEMARA®), and anastrozole) (ARIMIDEX®).

Examples of anti-androgens include apalutamide, abiraterone, enzalutamide, flutamide, galeterone, nilutamide, bicalutamide, leuprolide, goserelin, ODM-201, APC-100, ODM-204.

Examples of progesterone receptor antagonist include onapristone.

### Anti-Angiogenic Agents

Anti-angiogenic agents include, but are not limited to, retinoid acid and derivatives thereof, 2-methoxyestradiol, ANGIOSTATIN®, ENDOSTATIN®, regorafenib, necuparanib, suramin, squalamine, tissue inhibitor of metalloproteinase-1, tissue inhibitor of metalloproteinase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs such as 1-azetidine-2-carboxylic acid (LACA), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, metalloproteinase inhibitors such as BB-94, inhibitors of S100A9 such as tasquinimod. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2.

### Anti-Fibrotic Agents

Anti-fibrotic agents include, but are not limited to, the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. No. 5,021,456, U.S. Pat. No. 5,059,714, U.S. Pat. No. 5,120,764, U.S. Pat. No. 5,182,297, U.S. Pat. No. 5,252,608 relating to 2-(1-naphthyloxymemyl)-3-fluoroallylamine, and US 2004-0248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone.

Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethy)dithio)butanoic acid, p-2-amino-3-methyl-3-((2-aminoethy)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

### Immunotherapeutic Agents

The immunotherapeutic agents include and are not limited to therapeutic antibodies suitable for treating patients. Some examples of therapeutic antibodies include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, dinutuximab, ecromeximab, elotuzumab, emibetuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab (YERVOY®, MDX-010, BMS-734016, and MDX-101), iratumumab, labetuzumab, lexatumumab, lintuzumab, lorvotuzumab, lucatumumab, mapatumumab, matuzumab, milatuzumab, minretumomab, mitumomab, mogamulizumab, moxetumomab, pasudotox, narnatumab, naptumomab, necitumumab, nimotuzumab, nofetumomab, obinutuzumab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, ramucirumab (Cyramza®), rilotumumab, rituximab, robatumumab, samalizumab, satumomab, sibrotuzumab, siltuximab, solitomab, tacatuzumab, taplitumomab, tenatumomab, teprotumumab, tigatuzumab, tositumomab, trastuzumab, ABP-980, tucotuzumab, ubilituximab, veltuzumab, vorsetuzumab, votumumab, zalutumumab, CC49, OBI-833 and 3F8. Rituximab can be used for treating indolent B-cell cancers, including marginal-zone lymphoma, WM, CLL and small lymphocytic lymphoma. A combination of Rituximab and chemotherapy agents is especially effective.

The exemplified therapeutic antibodies may be further labeled or combined with a radioisotope particle such as indium-111, yttrium-90 (90Y-clivatuzumab), or iodine-131. Cancer Gene Therapy and Cell Therapy including the insertion of a normal gene into cancer cells to replace a mutated or altered gene; genetic modification to silence a mutated gene; genetic approaches to directly kill the cancer cells; including the infusion of immune cells designed to replace most of the patient's own immune system to enhance the immune response to cancer cells, or activate the patient's own immune system (T cells or Natural Killer cells) to kill cancer cells, or find and kill the cancer cells; genetic approaches to modify cellular activity to further alter endogenous immune responsiveness against cancer. Non limiting examples are Algenpantucel-L (2 pancreatic cell lines), Sipuleucel-T, SGT-53 liposomal nanodelivery (scL) of gene p53; T-cell therapy, such as CD19 CAR-T tisagenlecleucel-T (CTL019), KTE-C19, JCAR015, BXP-501; activated allogeneic natural killer cells CNDO-109-AANK, LFU-835 hematopoietic stem cells.

### Types of Cancer

Patients and cancers treated herein include Burkitt's lymphoma, Hodgkin's lymphoma, non-Hodgkin's lymphoma (NHL), indolent non-Hodgkin's lymphoma (iNHL), refractory iNHL, multiple myeloma (MM), chronic myeloid leukemia (CML), acute lymphocytic leukemia (ALL), B-cell ALL, acute myeloid leukemia (AML), chronic lymphocytic leukemia (CLL), small lymphocytic lymphoma (SLL), myelodysplastic syndrome (MDS), myeloproliferative disease (MPD), mantle cell lymphoma (MCL), follicular lymphoma (FL), Waldestrom's macroglobulinemia (WM), T-cell lymphoma, B-cell lymphoma, diffuse large B-cell lymphoma (DLBCL), or marginal zone lymphoma (MZL). In one embodiment, the cancer is minimal residual disease (MRD). In additional embodiment, the cancer is selected from Hodgkin's lymphoma, non-Hodgkin's lymphoma (NHL), indolent non-Hodgkin's lymphoma (iNHL), and refractory iNHL. In certain embodiment, the cancer is indolent non-Hodgkin's lymphoma (iNHL). In some embodiment, the cancer is refractory iNHL. In one embodiment, the cancer is chronic lymphocytic leukemia (CLL). In other embodiment, the cancer is diffuse large B-cell lymphoma (DLBCL).

In certain embodiments, the cancer is a solid tumor is selected from the group consisting of pancreatic cancer; bladder cancer; colorectal cancer; breast cancer, including metastatic breast cancer; prostate cancer, including androgen-dependent and androgen-independent prostate cancer; kidney or renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma, hepatic carcinoma, rectal cancer, penile carcinoma, vulval cancer, thyroid cancer, salivary gland carcinoma, endometrial or uterine carcinoma, hepatoma, hepatocellular cancer, liver cancer, gastric or stomach cancer including gastrointestinal cancer, cancer of the peritoneum, squamous carcinoma of the lung, gastroesophagal cancer, biliary tract cancer, gall bladder cancer, colorectal/appendiceal cancer, squamous cell cancer (e.g., epithelial squamous cell cancer).

Any of the methods of treatment provided may be used to treat cancer at various stages. By way of example, the cancer stage includes but is not limited to early, advanced, locally advanced, remission, refractory, reoccurred after remission and progressive.

Subjects

Any of the methods of treatment provided may be used to treat a subject (e.g., human) who has been diagnosed with or is suspected of having cancer. As used herein, a subject refers to a mammal, including, for example, a human.

In some embodiments, the subject may be a human who exhibits one or more symptoms associated with cancer or hyperproliferative disease. In some embodiments, the subject may be a human who exhibits one or more symptoms associated with cancer. In some embodiments, the subject is at an early stage of a cancer. In other embodiments, the subject is at an advanced stage of cancer.

In certain, the subject may be a human who is at risk, or genetically or otherwise predisposed (e.g., risk factor) to developing cancer or hyperproliferative disease who has or has not been diagnosed. As used herein, an “at risk” subject is a subject who is at risk of developing cancer. The subject may or may not have detectable disease, and may or may not have displayed detectable disease prior to the treatment methods described herein. An at risk subject may have one or more so-called risk factors, which are measurable parameters that correlate with development of cancer, which are described herein. A subject having one or more of these risk factors has a higher probability of developing cancer than an individual without these risk factor(s). These risk factors may include, for example, age, sex, race, diet, history of previous disease, presence of precursor disease, genetic (e.g., hereditary) considerations, and environmental exposure. In some embodiments, the subjects at risk for cancer include, for example, those having relatives who have experienced the disease, and those whose risk is determined by analysis of genetic or biochemical markers.

In addition, the subject may be a human who is undergoing one or more standard therapies, such as chemotherapy, radiotherapy, immunotherapy, surgery, or combination thereof. Accordingly, one or more kinase inhibitors may be administered before, during, or after administration of chemotherapy, radiotherapy, immunotherapy, surgery or combination thereof.

In certain embodiments, the subject may be a human who is (i) substantially refractory to at least one chemotherapy treatment, or (ii) is in relapse after treatment with chemotherapy, or both (i) and (ii). In some of embodiments, the subject is refractory to at least two, at least three, or at least four chemotherapy treatments (including standard or experimental chemotherapies).

### Lymphoma or Leukemia Combination Therapy

Some anticancer agents are suitable for treating lymphoma or leukemia. These agents include aldesleukin, alvocidib, antineoplaston AS2-1, antineoplaston A10, anti-thymocyte globulin, amifostine trihydrate, aminocamptothecin, arsenic trioxide, beta alethine, Bcl-2 family protein inhibitor ABT-263, venetoclax (ABT-199), BMS-345541, bortezomib) (VELCADE®), carfilzomib (Kyprolis®), vemurafenib (Zelboraf®), Omr-IgG-am (WHIG, Omrix), bryostatin 1, busulfan, carboplatin, campath-1H, CC-5103, carmustine, caspofungin acetate, clofarabine, cisplatin, cladribine, chlorambucil, curcumin, cyclosporine, cyclophosphamide, cytarabine, denileukin diftitox, dexamethasone, DT-PACE (dexamethasone, thalidomide, cisplatin, doxorubicin, cyclophosphamide, and etoposide), docetaxel, dolastatin 10, doxorubicin, doxorubicin hydrochloride, enzastaurin, epoetin alfa, etoposide, everolimus (RAD001), fenretinide, filgrastim, melphalan, mesna, flavopiridol, fludarabine, geldanamycin (17-AAG), ifosfamide, irinotecan hydrochloride, ixabepilone, lenalidomide (REVLIMID®, CC-5013), lymphokine-activated killer cells, melphalan, methotrexate, mitoxantrone hydrochloride, motexafin gadolinium, mycophenolate mofetil, nelarabine, oblimersen, obatoclax (GX15-070), oblimersen, octreotide acetate, omega-3 fatty acids, oxaliplatin, paclitaxel, palbociclib (PD0332991), PEGylated liposomal doxorubicin hydrochloride, pegfilgrastim, pentostatin, perifosine, prednisolone, prednisone, R-roscovitine (seliciclib, CYC202), recombinant interferon alfa, interferon alpha-2b, recombinant interleukin-12, recombinant interleukin-11, recombinant flt3 ligand, recombinant human thrombopoietin, rituximab, sargramostim, sildenafil citrate, simvastatin, sirolimus, styryl sulphones, tacrolimus, tanespimycin, temsirolimus (CC1-779), thalidomide, therapeutic allogeneic lymphocytes, thiotepa, tipifarnib, bortezomib (VELCADE®, PS-341), vincristine, vincristine sulfate, vinorelbine ditartrate, SAHA (suberanilohydroxamic acid, or suberoyl, anilide, and hydroxamic acid), FR (fludarabine and rituximab), CHOP (cyclophosphamide, doxorubicin, vincristine, and prednisone), CVP (cyclophosphamide, vincristine, and prednisone), FCM (fludarabine, cyclophosphamide, and mitoxantrone), FCR (fludarabine, cyclophosphamide, and rituximab), hyperCVAD (hyperfractionated cyclophosphamide, vincristine, doxorubicin, dexamethasone, methotrexate, and cytarabine), ICE (iphosphamide, carboplatin, and etoposide), MCP (mitoxantrone, chlorambucil, and prednisolone), R-CHOP (rituximab and CHOP), R-CVP (rituximab and CVP), R-FCM (rituximab and FCM), R-ICE (rituximab and ICE), and R-MCP (rituximab and MCP).

One modified approach is radioimmunotherapy, wherein a monoclonal antibody is combined with a radioisotope particle, such as indium-111, yttrium-90, and iodine-131. Examples of combination therapies include, but are not limited to, iodine-131 tositumomab) (BEXXAR®), yttrium-90 ibritumomab tiuxetan (ZEVALIN®), and BEXXAR® with CHOP.

The abovementioned therapies can be supplemented or combined with stem cell transplantation or treatment. Therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, low-LET cobalt-60 gamma ray therapy, bleomycin, conventional surgery, radiation therapy, and nonmyeloablative allogeneic hematopoietic stem cell transplantation.

### Non Hodgkin's Lymphomas Combination Therapy

Treatment of non-Hodgkin's lymphomas (NHL), especially those of B cell origin, includes using monoclonal antibodies, standard chemotherapy approaches (e.g., CHOP, CVP, FCM, MCP, and the like), radioimmunotherapy, and combinations thereof, especially integration of an antibody therapy with chemotherapy.

Examples of unconjugated monoclonal antibodies for the treatment of NHL/B-cell cancers include rituximab, alemtuzumab, human or humanized anti-CD20 antibodies, lumiliximab, anti-TNF-related apoptosis-inducing ligand (anti-TRAIL), bevacizumab, galiximab, epratuzumab, SGN-40, and anti-CD74.

Examples of experimental antibody agents used in treatment of NHL/B-cell cancers include ofatumumab, ha20, PRO131921, alemtuzumab, galiximab, SGN-40, CHIR-12.12, epratuzumab, lumiliximab, apolizumab, milatuzumab, and bevacizumab.

Examples of standard regimens of chemotherapy for NHL/B-cell cancers include CHOP, FCM, CVP, MCP, R-CHOP, R-FCM, R-CVP, and R-MCP.

Examples of radioimmunotherapy for NHL/B-cell cancers include yttrium-90 ibritumomab tiuxetan (ZEVALIN®) and iodine-131 tositumomab) (BEXXAR®).

### Mantle Cell Lymphoma Combination Therapy

Therapeutic treatments for mantle cell lymphoma (MCL) include combination chemotherapies such as CHOP, hyperCVAD, and FCM. These regimens can also be supplemented with the monoclonal antibody rituximab to form combination therapies R-CHOP, hyperCVAD-R, and R-FCM. Any of the abovementioned therapies may be combined with stem cell transplantation or ICE in order to treat MCL.

An alternative approach to treating MCL is immunotherapy. One immunotherapy uses monoclonal antibodies like rituximab. Another uses cancer vaccines, such as GTOP-99, which are based on the genetic makeup of an individual patient's tumor.

A modified approach to treat MCL is radioimmunotherapy, wherein a monoclonal antibody is combined with a radioisotope particle, such as iodine-131 tositumomab (BEXXAR®) and yttrium-90 ibritumomab tiuxetan) (ZEVALIN®). In another example, BEXXAR® is used in sequential treatment with CHOP.

Other approaches to treating MCL include autologous stem cell transplantation coupled with high-dose chemotherapy, administering proteasome inhibitors such as bortezomib (VELCADE® or PS-341), or administering antiangiogenesis agents such as thalidomide, especially in combination with rituximab.

Another treatment approach is administering drugs that lead to the degradation of Bcl-2 protein and increase cancer cell sensitivity to chemotherapy, such as oblimersen, in combination with other chemotherapeutic agents.

A further treatment approach includes administering mTOR inhibitors, which can lead to inhibition of cell growth and even cell death. Non-limiting examples are sirolimus, temsirolimus (TORISEL®, CCI-779), CC-115, CC-223, SF-1126, PQR-309, voxtalisib, GSK-2126458, and temsirolimus in combination with RITUXAN®, VELCADE®, or other chemotherapeutic agents.

Other recent therapies for MCL have been disclosed. Such examples include flavopiridol, palbociclib (PD0332991), R-roscovitine (selicicilib, CYC202), styryl sulphones, obatoclax (GX15-070), TRAIL, Anti-TRAIL death receptors DR4 and DR5 antibodies, temsirolimus (TORISEL®, CC1-779), everolimus (RAD001), BMS-345541, curcumin, SAHA, thalidomide, lenalidomide (REVLIMID®, CC-5013), and geldanamycin (17-AAG).

### Waldenstrom's Macroglobulinemia Combination Therapy

Therapeutic agents used to treat Waldenstrom's Macroglobulinemia (WM) include perifosine, bortezomib) (VELCADE®), rituximab, CC-5103, thalidomide, epratuzumab (hLL2-anti-CD22 humanized antibody), simvastatin, enzastaurin, campath-1H, dexamethasone, DT-PACE, oblimersen, antineoplaston A10, antineoplaston AS2-1, alemtuzumab, beta alethine, cyclophosphamide, doxorubicin hydrochloride, prednisone, vincristine sulfate, fludarabine, filgrastim, melphalan, recombinant interferon alfa, carmustine, cisplatin, cyclophosphamide, cytarabine, etoposide, melphalan, dolastatin 10, indium-111 monoclonal antibody MN-14, yttrium-90 humanized epratuzumab, anti-thymocyte globulin, busulfan, cyclosporine, methotrexate, mycophenolate mofetil, therapeutic allogeneic lymphocytes, yttrium-90 ibritumomab tiuxetan, sirolimus, tacrolimus, carboplatin, thiotepa, paclitaxel, aldesleukin, docetaxel, ifosfamide, mesna, recombinant interleukin-11, recombinant interleukin-12, Bcl-2 family protein inhibitor ABT-263, denileukin diftitox, tanespimycin, everolimus, pegfilgrastim, vorinostat, alvocidib, recombinant flt3 ligand, recombinant human thrombopoietin, lymphokine-activated killer cells, amifostine trihydrate, aminocamptothecin, irinotecan hydrochloride, caspofungin acetate, clofarabine, epoetin alfa, nelarabine, pentostatin, sargramostim, vinorelbine ditartrate, WT-1 analog peptide vaccine, WT1 126-134 peptide vaccine, fenretinide, ixabepilone, oxaliplatin, monoclonal antibody CD19 (such as tisagenlecleucel-T, CART-19, CTL-019), monoclonal antibody CD20, omega-3 fatty acids, mitoxantrone hydrochloride, octreotide acetate, tositumomab, iodine-131 tositumomab, motexafin gadolinium, arsenic trioxide, tipifarnib, autologous human tumor-derived HSPPC-96, veltuzumab, bryostatin 1, PEGylated liposomal doxorubicin hydrochloride, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme techniques, low-LET cobalt-60 gamma ray therapy, bleomycin, conventional surgery, radiation therapy, and nonmyeloablative allogeneic hematopoietic stem cell transplantation.

### Diffuse Large B-Cell Lymphoma Combination Therapy

Therapeutic agents used to treat diffuse large B-cell lymphoma (DLBCL) include cyclophosphamide, doxorubicin, vincristine, prednisone, anti-CD20 monoclonal antibodies, etoposide, bleomycin, many of the agents listed for WM, and any combination thereof, such as ICE and R-ICE.

### Chronic Lymphocytic Leukemia Combination Therapy

Examples of therapeutic agents used to treat chronic lymphocytic leukemia (CLL) include chlorambucil, cyclophosphamide, fludarabine, pentostatin, cladribine, doxorubicin, vincristine, prednisone, prednisolone, alemtuzumab, many of the agents listed for WM, and combination chemotherapy and chemoimmunotherapy, including the following common combination regimens: CVP, R-CVP, ICE, R-ICE, FCR, and FR.

### Myelofibrosis Combination Therapy

Myelofibrosis inhibiting agents include, but are not limited to, hedgehog inhibitors, histone deacetylase (HDAC) inhibitors, and tyrosine kinase inhibitors. Non-limiting examples of hedgehog inhibitors are saridegib and vismodegib.

Examples of HDAC inhibitors include, but are not limited to, pracinostat and panobinostat.

Non-limiting examples of tyrosine kinase inhibitors are lestaurtinib, bosutinib, imatinib, gilteritinib, radotinib, and cabozantinib.

### Hyperproliferative Disorder Combination Therapy

Gemcitabine, nab-paclitaxel, and gemcitabine/nab-paclitaxel may be used with a JAK inhibitor and/or PI3δ inhibitor to treat hyperproliferative disorders.

In the following description of the examples, specific embodiments in which the invention may be practiced are described. These embodiments are described in sufficient detail to enable those skilled in the art to practice the invention. Other embodiments may be utilized, and logical and other changes may be made without departing from the scope of the invention. The following detailed description is, therefore, not to be taken in a limiting sense, and the scope of the invention is defined only by the appended claims, along with the full scope of equivalents to which such claims are entitled.

It is understood that cancer cells expressing CYP3A enzymes may be a result of the pathology of the cancer and/or a result of administration/contact with an anticancer agent (i.e. not caused by cancer per se, but the treatment) due to increased stress to the cell.

One embodiment of the invention provides a method for treating a patient suffering from cancer comprising administering to said patient: (a) an anticancer agent; and (b) cobicistat; wherein, the cancer comprises cells expressing a CYP3A enzyme and the concentration of the anticancer agent in the cells is increased after administration of cobicistat. In another embodiment, the cancer comprises cells overexpressing a CYP3A enzyme.

Another embodiment provides a method for enhancing the effect of an anticancer agent in a patient suffering from cancer comprising administering to said patient: (a) the anticancer agent; and (b) cobicistat; wherein, the cancer comprises cells expressing a CYP3A enzyme and the effect of the anticancer agent in the cells is increased after administration of cobicistat. In another embodiment, the cancer comprises cells overexpressing a CYP3A enzyme.

Another embodiment provides a method for reducing metabolism of an anticancer agent in a patient suffering from cancer comprising administering to said patient: (a) the anticancer agent; and (b) cobicistat; wherein, the cancer comprises cells expressing a CYP3A enzyme and the metabolism of the anticancer agent in the cells is decreased after administration of cobicistat. In another embodiment, the cancer comprises cells that overexpress the CYP3A enzyme.

Another embodiment of the invention provides for a method for increasing sensitivity to an anticancer agent in a patient suffering from cancer comprising administering to said patient: (a) the anticancer agent; and (b) cobicistat; wherein, cobicistat increases sensitivity to the anticancer agent. In another embodiment, the increase is by at least 2-fold, or 1.5-fold, or 3-fold or 5-fold. More particularly, 2-fold. In another embodiment, the cancer comprises cells overexpressing a CYP3A enzyme.

In another embodiment, the CYP3A enzyme is CYP3A4. In another embodiment, the cancer is liver, pancreatic, breast, kidney, colon, lung, uterine, bladder, thyoma, prostate, thyroid, bladder, esophageal, cervical, sarcoma, or a cancer comprising cell lines expressing gain-of-function mutations in TP53.

In another embodiment, the CYP3A enzyme is CYP3A5. In another embodiment, the cancer is breast, pancreatic, thyroid, kidney, cervical or skin.

In another embodiment, the anticancer agent is selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids and ZD1839; or a pharmaceutically acceptable salt thereof.

In another embodiment, the anticancer agent is docetaxel. In another embodiment, the anticancer agent is paclitaxel.

In another embodiment, cobicistat and the anticancer agent are administered to the patient in separate dosage forms. In another embodiment, cobicistat and the anticancer agent are administered to the patient as a fixed-dose combination.

In another embodiment, cobicistat is administered to the patient once a day. In another embodiment, cobicistat is administered to the patient twice a day. In another embodiment, cobicistat is administered to the patient once every other a day.

In another embodiment, the therapeutic index (TI) of the anticancer agent is greater than 1, or 1.1, or 1.2, or 1.3, or 1.4, or 1.5, or 1.6, or 1.7, or 1.8, or 1.9, or 2 or 2.5, or 3, or 4, or 5.

In another embodiment, the patient is not being treated for HIV.

Another embodiment provides a pharmaceutical composition comprising (a) an anticancer agent; (b) cobicistat; and (c) a carrier.

Another embodiment provides for the use of: (a) an anticancer agent; and (b) cobicistat; for treating a patient suffering from cancer, the cancer comprises cells expressing a CYP3A enzyme and the concentration of the anticancer agent in the cells is increased after administration of cobicistat. Another embodiment provides for the use of: (a) an anticancer agent; and (b) cobicistat; in the manufacture of a medicament for treating a patient suffering from cancer, the cancer comprises cells expressing a CYP3A enzyme and the concentration of the anticancer agent in the cells is increased after administration of cobicistat. Another embodiment provides for the use of: (a) the anticancer agent; and (b) cobicistat; for increasing sensitivity to an anticancer agent in a patient suffering from cancer. In another embodiment, cobicistat increases sensitivity to the anticancer agent by at least 2-fold. Another embodiment provides for use of: (a) the anticancer agent; and (b) cobicistat; for reducing metabolism of an anticancer agent.

Another embodiment provides for the use of: (a) an anticancer agent; and (b) cobicistat; for enhancing the effect of the anticancer agent in a patient suffering from cancer comprising administering to said patient wherein, the cancer comprises cells expressing a CYP3A enzyme and the effect of the anticancer agent in the cells is increased after administration of cobicistat. Another embodiment provides for the use of: (a) an anticancer agent; and (b) cobicistat; in the manufacture of a medicament for enhancing the effect of the anticancer agent in a patient suffering from cancer comprising administering to said patient wherein, the cancer comprises cells expressing a CYP3A enzyme and the effect of the anticancer agent in the cells is increased after administration of cobicistat. Another embodiment provides for the use of: (a) the anticancer agent; and (b) cobicistat; in the manufacture of a medicament for increasing sensitivity to an anticancer agent in a patient suffering from cancer. In another embodiment, cobicistat increases sensitivity to the anticancer agent by at least 2-fold. Another embodiment provides for use of: (a) the anticancer agent; and (b) cobicistat; in the manufacture of a medicament for reducing metabolism of an anticancer agent.

Cytochromes P450 (CYPs) are key enzymes involved in drug metabolism in normal physiologic conditions. Their activity has been used in drug development as CYPs can activate certain pro-drugs resulting in effective agents. CYPs also metabolize drugs into inactive forms. In certain cancer cell lines, CYPs are expressed in their basal state or in response to cellular stress, thereby having a pronounced effect on drugs targeting particular cell lines. This expression can be an intrinsic property of the tumour or be induced upon therapeutic treatment. In particular, CYP3A5 has been shown to be expressed and induced in different subtypes of pancreatic ductal adenocarcinoma (PDAC) cells, resulting in lack or diminished sensitivity to several chemotherapeutic agents. Noll et al., Nat. Medicine, v. 22(3), March 2016. It is been shown that drug resistance to PDAC results from accelerated metabolism of anticancer agents targeting the cells.

Targeting CYP activity is challenging and can result in greater toxicity to patients already suffering from debilitating diseases. This is especially true for non-specific CYP inhibitors or agents that inhibit CYPs involved in metabolism of endogenous compounds (fatty acids, vitamins, steroids etc.). Because cancer patients often take a variety of different drugs, many of which are CYP substrates, selectivity in inhibition is important for treatment. Additionally, certain CYPs are expressed in a highly tissue-specific, restricted manner. Targeting these CYPS may result in reduced systemic toxicity.

At present more than 57 active human P450 genes and 58 pseudogenes are known. Rodriguez-Antona et al. Oncogene (2006) 25, 1679-1691. The most polymorphic CYPs are on the CYP2B6 (48 alleles), CYP2C9 (32), CYP2D6 (92) and CYP3A4 (34). Most of the functional polymorphisms are seen regarding the variability in the CYP2A6, CYP2B6, CYP2C9, CYP2C19 and CYP2D6 genes. Therefore, finding a single agent that is selective, yet potent and effective, at inhibiting a specific CYP, is difficult.

### EXAMPLES

### Example 1

### Cell Preparation.

All cell lines were obtained from the American Type Culture Collection (ATCC) Manassas, Va. (US). Master and working cell banks (MCB and WCB) were prepared by subculturing in ATCC-recommended media and freezing protocols (www.atcc.org). Cell line stocks for the assays were prepared from the WCB. The MCB, WCBs and assay stocks were prepared within respectively 3, 6 and 9 passages of the ATCC vial.

### Compound Preparation.

Solid powders of reference compounds were weighed on a calibrated balance and dissolved in 100% DMSO. DMSO samples were stored at room temperature. At the day of the experiment, the compound stock was diluted in 3.16 fold steps in 100% DMSO to obtain a 9-point dilution series. This was further diluted 31.6 times in 20 mM sterile Hepes buffer pH 7.4. A volume of 5 μl was transferred to the cells to generate a test concentration range from 3.16×10⁻⁵M to 3.16×10⁻⁹ M in duplicate. The final DMSO concentration during incubation was 0.4% in all wells. If a compound showed very potent activity, the testing range was expanded to ensure a full dose-response curve could be measured in duplicate.

### Cell Proliferation Assay.

An assay stock was thawed and diluted in its ATCC recommended medium and dispensed in a 384-well plate, depending on the cell line used, at a concentration of 200-3200 cells per well in 45 μl medium. For each used cell line the optimal cell density is used. The margins of the plate were filled with phosphate-buffered saline. Plated cells were incubated in a humidified atmosphere of 5% CO₂ at 37° C. After 24 hours, 5 μl of compound dilution was added and plates were further incubated for another 120 hours. After 120 hours, 25 μl of ATPlite 1Step™ (PerkinElmer) solution was added to each well, and subsequently shaken for 2 minutes. After 10 minutes of incubation in the dark, the luminescence was recorded on an Envision multimode reader (PerkinElmer).

### Controls

### T=0 Signal.

On a parallel plate, 45 μl cells were dispensed and incubated in a humidified atmosphere of 5% CO₂ at 37° C. After 24 hours 5 μl DMSO-containing Hepes buffer and 25 μl ATPlite 1Step™ solution were mixed, and luminescence measured after 10 minutes incubation (=luminescenceₜ₋₀).

### Reference Compound.

The IC₅₀ of the reference compound doxorubicin is measured on a separate plate. The IC₅₀ is trended. If the IC₅₀ is out of specification (0.32-3.16 times deviating from historic average) the assay is invalidated.

### Cell Growth Control.

The cellular doubling times of all cell lines are calculated from the t=0 hours and t=120 hours growth signals of the untreated cells. If the doubling time is out of specification (0.5-2.0 times deviating from historic average) the assay is invalidated.

### Maximum Signals.

For each cell line, the maximum luminescence was recorded after incubation for 120 hours without compound in the presence of 0.4% DMSO (=luminescence_{untreated,t=120h}).

### Data Analysis

IC₅₀s were calculated by non-linear regression using IDBS XLfit 5. The percentage growth after 120 h (%-growth) was calculated as follows: 100%×(luminescenceₜ₌₁₂₀ₕ/luminescence_{untreated,t=120h}). This was fitted to the ¹⁰log compound concentration (conc) by a 4-parameter logistics curve: %−growth=bottom+(top−bottom)/(1+10^{(log IC50-conc)*hill)}), where hill is the Hill-coefficient, and bottom and top the asymptotic minimum and maximum cell growth that the compound allows in that assay.

### NCI60 Parameters

The LD₅₀, the concentration at which 50% of cells die, is the concentration where luminescenceₜ₌₁₂₀ₕ=½×luminescenceₜ₌₀ₕ. The GI₅₀, the concentration of 50% growth inhibition, is the concentration where cell growth is half maximum. This is concentration associated with the signal: ((luminescence_{untreated,t=120h}−luminescenceₜ₌₀)/2)+luminescenceₜ₌₀. R. H. Shoemaker (2006), Nature Reviews Cancer 6: 814-823.

### Drug Sensitivity.

The ¹⁰log IC₅₀ differences between the “modified and “wild type’ groups of cell lines were analyzed in three ways. First, for the eighteen most frequent genetic changes, drug sensitivities of individual cell lines were visualized in waterfall. Secondly, a larger subset of the most commonly occurring and best known cancer genes (38 in total) was analyzed with type II Anova analysis in the statistical program R. Thirdly, the complete set of 114 cancer genes was analyzed by a two-sided homoscedastic t-test in R.

The p-values from Anova and t-test were subjected to a Benjamini-Hochberg multiple testing correction, and only genetic associations with a false discovery rate less than 20% are considered significant. Results of the 38-gene analysis were visualized in a volcano plot. The type II Anova analysis on 38 cancer genes is a different test than the homoscedastic t-test on 114 cancer genes, meaning that the significance of the associations may differ. For Additional information on Oncolines™ is described in J. C. M. Uitdehaag et al. (2014), PLoS ONE 9: e92146.

### Results:

The results of the studies are reflected in Table 1.

TABLE 1

| | Fold change in IC50 (IC50 [Drug + | | | | | |
|---|---|---|---|---|---|---|
| | Cobicistat]/IC50 [Drug]) | | | | | |
| Cell line | dasatinib | docetaxel | doxorubicin | paclitaxel | vinblastine | vincristine |
| 769-P | 3.8 | 3.0 | 5.1 | 2.9 | 2.5 | 3.5 |
| 786-O | 1.6 | 4.2 | 2.3 | 3.0 | 2.5 | 2.5 |
| A-172 | 1.7 | 2.0 | 2.5 | 1.7 | 3.0 | 3.3 |
| A-204 | 2.2 | 5.7 | 2.5 | 3.6 | 4.0 | 4.4 |
| A375 | 1.3 | 2.8 | 2.2 | 2.3 | 3.4 | 4.4 |
| A388 | 1.9 | 6.0 | 1.7 | 2.7 | 4.7 | 3.7 |
| A-427 | 5.8 | 1.3 | 1.5 | 1.2 | 2.7 | 1.9 |
| A-498 | 1.4 | 4.2 | 1.2 | 2.8 | 3.8 | 3.2 |
| A-549 | 1.0 | 1.1 | 0.8 | 1.0 | 2.3 | 1.9 |
| A-704 | 1.0 | 2.3 | 1.3 | 1.5 | 3.8 | 2.0 |
| ACHN | 2.8 | 4.6 | 2.2 | 2.5 | 3.8 | 3.4 |
| AN3 CA | 1.2 | 2.1 | 2.0 | 2.9 | 3.9 | 4.7 |
| AsPC-1 | 1.2 | 5.0 | 1.3 | 2.2 | 5.6 | 3.1 |
| AU-565 | 8.4 | 3.2 | 2.1 | 2.7 | 3.0 | 3.0 |
| BT-20 | 1.4 | 1.7 | 1.0 | 1.5 | 2.7 | 3.2 |
| BT-549 | 1.5 | 1.4 | 1.5 | 1.2 | 3.3 | 2.9 |
| BxPC-3 | 2.2 | 3.0 | 2.2 | 3.2 | 2.4 | 4.1 |
| C-33 A | 1.2 | 1.5 | 1.9 | 1.3 | 1.9 | 2.7 |
| CAL 27 | 1.3 | 1.4 | 1.9 | 2.0 | 2.8 | 2.6 |
| CCRF-CEM | 1.3 | 3.6 | 2.7 | 2.6 | 3.3 | 3.6 |
| COLO 205 | 1.2 | 3.9 | 2.3 | 3.0 | 4.9 | 4.0 |
| COLO 829 | 0.0 | 3.7 | 3.8 | 4.1 | 6.3 | 4.4 |
| Daoy | 3.2 | 6.4 | 1.6 | 3.4 | 5.7 | 4.6 |
| DLD-1 | 1.4 | 2.7 | 1.4 | 1.9 | 2.2 | 2.0 |
| DoTc2 4510 | 3.0 | 5.3 | 1.7 | 3.4 | 5.0 | 6.2 |
| DU 145 | 3.6 | 4.8 | 2.1 | 4.8 | 5.4 | 3.8 |
| FaDu | 2.1 | 1.7 | 1.7 | 1.3 | 2.2 | 2.8 |
| HCT 116 | 3.4 | 1.9 | 1.8 | 1.0 | 2.0 | 1.4 |
| HCT-15 | 2.6 | 2.8 | 2.0 | 2.4 | 2.3 | 2.1 |
| Hs 578T | 2.5 | 1.9 | 1.7 | 2.6 | 3.7 | 2.8 |
| J82 | 2.1 | 3.5 | 2.2 | 2.9 | 4.2 | 3.6 |
| Jurkat E6.1 | NA* | 2.2 | 2.5 | 2.5 | 2.5 | 2.8 |
| K-562 | 1.1 | 3.2 | 2.5 | 2.4 | 2.6 | 3.5 |
| KU812 | 1.5 | 5.6 | 2.9 | 4.6 | 4.4 | 3.7 |
| LNCaP | 1.3 | 3.8 | 1.8 | 2.5 | 4.0 | 3.6 |
| FGC | | | | | | |
| LoVo | 1.0 | 2.0 | 2.0 | 1.3 | 1.9 | 1.6 |
| LS 174T | NA | 2.8 | 3.6 | 1.8 | 3.4 | 2.5 |
| MCF7 | 1.9 | 4.1 | 1.9 | 3.3 | 5.3 | 4.8 |
| MeWo | 1.0 | 2.3 | 2.1 | 2.0 | 3.2 | 2.9 |
| MG-63 | 2.9 | 3.3 | 2.4 | 2.4 | 2.8 | 3.8 |
| MIA PaCa-2 | 2.2 | 4.2 | 2.2 | 3.0 | 3.7 | 3.6 |
| MOLT-4 | 1.1 | 1.7 | 1.9 | 2.1 | 2.8 | 2.1 |
| NCI-H460 | NA | 2.1 | 2.0 | 2.0 | 3.4 | 3.1 |
| NCI-H82 | 1.1 | 2.2 | 2.6 | 2.4 | 3.5 | 3.1 |
| OVCAR-3 | 1.6 | 2.2 | 2.5 | 1.7 | 2.6 | 4.0 |
| PA-1 | 2.6 | 5.2 | 3.0 | 3.9 | 6.3 | 4.2 |
| RKO | NA | 3.8 | 2.9 | 3.3 | 3.6 | 3.7 |
| RPMI-7951 | 3.0 | 3.3 | 2.3 | 2.9 | 3.2 | 4.6 |
| RT4 | 2.5 | 4.4 | 2.7 | 3.7 | 4.9 | 4.0 |
| SHP-77 | 1.4 | 3.5 | 2.5 | 2.3 | 2.0 | 2.6 |
| SJCRH30 | 1.4 | 4.1 | 2.2 | 1.6 | 3.8 | 3.5 |
| SK-N-AS | 1.6 | 3.3 | 2.3 | 2.3 | 3.7 | 4.7 |
| SK-N-FI | 2.0 | 5.1 | 2.0 | 2.5 | 4.5 | 2.7 |
| SNU-C2B | 0.9 | 4.8 | 1.2 | 3.0 | 4.5 | 2.4 |
| SR | 1.9 | 3.6 | 1.8 | 1.8 | 4.0 | 3.6 |
| SUP-T1 | 1.4 | 4.5 | 2.0 | 3.5 | 3.0 | 3.6 |
| SW48 | 2.2 | 1.6 | 1.8 | 1.4 | 2.5 | 2.7 |
| SW480 | 1.0 | 2.3 | 2.5 | 1.9 | 3.3 | 3.3 |
| SW620 | 3.2 | 2.7 | 2.6 | 1.8 | 2.7 | 3.0 |
| SW948 | 1.2 | 3.5 | 2.5 | 2.6 | 4.2 | 3.5 |
| T24 | 3.9 | 6.3 | 1.8 | 4.4 | 5.2 | 4.4 |
| T98G | 2.3 | 7.7 | 1.4 | 5.2 | 4.9 | 4.1 |
| TT | 0.8 | 3.6 | 1.1 | 3.0 | 8.7 | 4.8 |
| U-2 OS | 4.0 | 4.3 | 3.0 | 3.2 | 4.4 | 3.5 |
| U-87 MG | 1.1 | 2.0 | 0.7 | 2.2 | 2.6 | 3.1 |
| VA-ES-BJ | 3.1 | 5.0 | 2.8 | 2.8 | 4.3 | 5.4 |

NA: Data did not produce a measurable IC50 to generate a comparison between treatments.

Co-administration of cobicistat broadly increased the sensitivity of many cell lines tested to the primary drug, compared to primary drug alone. On average, across all experiments, cobicistat increased sensitivity by 2.9-fold, with a maximum of 8.7-fold. The highest average increase in sensitivity was seen in cancer cells of the bladder, bone and prostate. Broad activity was also seen in cancer cells from blood, central nervous system, breast, colon and skin. Docetaxel, vinblastine and vincristine were boosted to the greatest extent.

The contents of the articles, patents and references cited herein are incorporated by reference.

1. A method for treating a patient suffering from cancer comprising administering to said patient: (a) an anticancer agent; and (b) cobicistat; wherein, the cancer comprises cells expressing a CYP3A enzyme and the concentration of the anticancer agent in the cells is increased after administration of cobicistat.

2. A method for enhancing the effect of an anticancer agent in a patient suffering from cancer comprising administering to said patient: (a) the anticancer agent; and (b) cobicistat; wherein, the cancer comprises cells expressing a CYP3A enzyme and the effect of the anticancer agent in the cells is increased after administration of cobicistat.

3. (canceled)

4. A method for reducing metabolism of an anticancer agent in a patient suffering from cancer comprising administering to said patient: (a) the anticancer agent; and (b) cobicistat; wherein, the cancer comprises cells expressing a CYP3A enzyme and the metabolism of the anticancer agent in the cells is decreased after administration of cobicistat.

5. The method of claim 4, wherein the cancer comprises cells that overexpress the CYP3A enzyme.

6. The method of claim 1, wherein the CYP3A enzyme is CYP3A4.

7. The method of claim 1, wherein the CYP3A enzyme is CYP3A5.

8. The method of claim 1, wherein the cancer is liver, pancreatic, breast, kidney, colon, lung, uterine, bladder, thyoma, prostate, thyroid, bladder, esophageal, cervical, sarcoma, or a cancer comprising cell lines expressing gain-of-function mutations in TP53.

9. The method of claim 8, wherein the cancer is breast, pancreatic, thyroid, kidney, cervical or skin.

10. The method of claim 1, wherein the anticancer agent is selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids and ZD1839; or a pharmaceutically acceptable salt thereof.

11. The method of claim 1, wherein the anticancer agent is selected from the group consisting of docetaxel, vinblastine and vincristine; or a pharmaceutically acceptable salt thereof.

12. The method of claim 1, wherein cobicistat and the anticancer agent are administered to the patient in separate dosage forms.

13. The method of claim 1, wherein cobicistat is administered to the patient once a day.

14. The method of claim 1, wherein cobicistat and the anticancer agent are administered to the patient in a fixed dose combination.

15. The method of claim 1, wherein the therapeutic index (TI) of the anticancer agent is greater than 1.

16. The method of claim 1, wherein the TI of the anticancer agent is greater than 2.

17. The method of claim 1, wherein the patient is not being treated for HIV.

18. (canceled)

19. The method of claim 2, wherein the anticancer agent is selected from the group consisting of docetaxel, vinblastine and vincristine; or a pharmaceutically acceptable salt thereof.

20. The method of claim 4, wherein the anticancer agent is selected from the group consisting of docetaxel, vinblastine and vincristine; or a pharmaceutically acceptable salt thereof.

21. The method of claim 2, wherein the patient is not being treated for HIV.

22. The method of claim 4, wherein the patient is not being treated for HIV.
